(19) United States
(12) Patent Application Publication
(10) Pub. No.: US 2023/0371969 A1
(43) Pub. Date: Nov. 23, 2023

(54) DEVICES, SYSTEMS, AND METHODS FOR TISSUE RETRACTION

(71) Applicant: BOSTON SCIENTIFIC SCIMED, INC., Maple Grove, MN (US)

(72) Inventors: Juan Pablo Ortiz Garcia, Heredia (CR); Barry Weitzner, Acton, MA (US); Yeison Calvo, San Ramón (CR); Rosa Angelica Perez Chaves, Heredia (CR); Julian Fuentes Castro, San José (CR); Jimena Cespedes Berrocal, Alajuela (CR); Cristian Araya Camacho, Tambor (CR)

(73) Assignee: BOSTON SCIENTIFIC SCIMED, INC., MAPLE GROVE, MN (US)

(21) Appl. No.: 18/198,128

(22) Filed: May 16, 2023

Related U.S. Application Data
(60) Provisional application No. 63/342,780, filed on May 17, 2022.

(51) Int. Cl. A61B 17/29

(52) U.S. Cl. CPC A61B 17/29; A61B 2017/2905; A61B 2017/00296

(57) ABSTRACT

A tissue traction device having a segment defined along a limited extent about the perimeter of the tissue traction device and more flexible than surrounding regions of the tissue traction device. The tissue traction device is actuatable to bend along the more flexible segment thereof, generally in a direction of a side of the tissue traction device opposite the more flexible segment thereof. The tissue traction device may be formed from one or more flexible tubular elements, with the flexible segment along a limited extent about the perimeter of the one or more tubular elements. A tissue traction system may be configured to provide actuation medium, such as an inflation medium, to the tissue traction device to control movement of the tissue traction device. The tissue traction system may include one or more flexible tubular elements, a distal end of which forms the tissue traction device.

### CROSS-REFERENCE TO RELATED APPLICATIONS

This application claims the benefit of priority under 35 U.S.C. § 119 to U.S. Provisional Application No. 63/342,780, filed May 17, 2022, the entire disclosure of which is hereby incorporated by reference herein for all purposes.

### FIELD

The present disclosure relates generally to the field of devices, systems, and methods for applying traction to tissue. More particularly, the present disclosure relates to devices, systems, and methods for applying traction to tissue for applying traction to tissue in a controllable direction, such as more than one direction.

### BACKGROUND

Various endoscopic surgical procedures require maneuvering about various anatomical structures. Some procedures, such as endoscopic mucosal resection (EMR), Endoscopic Submucosal Dissection (ESD), Pre-Oral Endoscopic Myotomy (POEM), etc., allow for minimally invasive endoscopic removal of benign and early malignant lesions, such as in the gastrointestinal (GI) tract. Minimally-invasive surgical techniques like these typically allow for faster recovery than with open or laparascopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the endoscope, occluding visibility of the camera and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Also, the elements used with such solutions may require separate medical tools than those used to perform the procedure, and such tools may even require a separate working channel in the endoscope, thereby potentially increasing the size and/or complexity of the endoscope. Alternative solutions for lifting tissue during a procedure which reduce cost, complexity, and cognitive load presented by currently available solutions would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a tissue traction system has a tissue traction device; and one or more elongate elements operatively coupled with the tissue traction device to actuate bending of the tissue traction device. In some aspects, a longitudinal circumferential region of the tissue traction device is more flexible than surrounding regions of the tissue traction device so that actuation of the tissue traction device causes bending along the more flexible region toward a less flexible region.

In some embodiments, the tissue traction device is formed by a distal extent of the one or more elongate elements extending a length proximal to distal ends of the one or more elongate elements. In some embodiments, at least one of the elongate elements is a flexible tubular element. In some embodiments, the at least one flexible tubular element has a sealed distal end. In some embodiments, the tissue traction system further includes an actuation mechanism operatively coupled to the one or more elongate elements to cause bending of the tissue traction device. In some embodiments, a distal extent of the at least one flexible tubular element forms a flexible tubular element of the tissue traction device; the longitudinal circumferential region of the tissue traction device is formed along a distal region of the at least one flexible tubular element and is more expandable than surrounding regions of the tissue traction device; and the actuation mechanism delivers an inflation medium through the at least one flexible tubular element to the tissue traction device to expand the longitudinal circumferential region of the tissue traction device relative to surrounding regions of the tissue traction device to cause the tissue traction device to bend.

In some embodiments, the tissue traction system further includes an actuation mechanism operatively coupled to the one or more elongate elements to cause bending of the tissue traction device.

In some embodiments, a stiffening element is applied along a limited region along the perimeter of the tissue traction device, resulting in surrounding regions being relatively more flexible.

In some embodiments, the one or more elongate elements includes one flexible tubular element with a sealed distal end; the tissue traction device is formed by a distal extent of the flexible tubular element extending a length proximal to the sealed distal end of the flexible tubular element; and the tissue traction device is directionally inflatable upon application of an inflation medium thereto causing the more flexible region of the tissue traction device to expand and to bend the tissue traction device toward the stiffening element.

In accordance with various principles of the present disclosure, a tissue traction device has an elongate cylindrical body extending along a longitudinal axis and having a distal end and a proximal end and a flexible segment therebetween extending along only a portion of the perimeter of the cylindrical body. In some aspects, the flexible segment is more flexible than surrounding portions of the cylindrical body to facilitate bending of the cylindrical body along the flexible segment.

In some embodiments, the cylindrical body is tubular and inflatable; and the flexible segment is more expandable than surrounding portions of the cylindrical body so that inflation of the tubular cylindrical body causes expansion of the flexible segment and bending of the flexible segment towards an opposite, less expandable side of the cylindrical body.

In some embodiments, the flexible segment extends only along a limited extent along the longitudinal axis of the cylindrical body.

In some embodiments, the cylindrical body is formed of a flexible material and a stiffening element is applied to a limited extent around the perimeter of the flexible cylindrical body, the flexible segment being defined opposite the stiffening element.

In some embodiments, the cylindrical body is a distal segment of a flexible tubular element of a tissue traction system.

In some aspects, a method for applying traction with a tissue traction device includes actuating the tissue traction device to bend along a flexible region of a grasping end thereof which is more flexible than surrounding regions, towards a region of the tissue traction device opposite the flexible region to cause the tissue traction device to apply traction via the grasping end thereof. In some aspects, the tissue traction device is tubular, and the method further includes actuating the tissue traction device by delivering an inflation medium to a lumen defined within the tissue traction device to expand the flexible segment of the tissue traction device relative to surrounding regions to cause the tissue traction device to bend in a direction generally opposite the flexible segment.

In some embodiments, the tissue traction device includes a plurality of flexible tubular elements coupled together along a common join region; the flexible segment of the tissue traction device is formed along a segment of each of the plurality of flexible tubular elements generally opposite the common join region; and the method includes selectively actuating one or more of the plurality of flexible tubular elements of the tissue traction device to control bending of the actuated flexible tubular element towards the common join region and to control bending of the tissue traction device in more than one direction. In some embodiments, each flexible tubular element is formed by a distal segment of a flexible tubular element forming a tissue traction system, each flexible tubular element having a lumen for delivering inflation medium to the tissue traction device and a sealed distal end, and the method further includes selectively delivering inflation medium through one or more of the flexible tubular elements to control bending of the tissue traction device in more than one direction.

In some embodiments, the flexible tubular element of the tissue traction device is formed along a distal segment of a flexible tubular element having a lumen for delivering inflation medium to the tissue traction device and a sealed distal end; a stiffening element is applied along a limited region about the perimeter of the flexible tubular element of the tissue traction device so that surrounding regions define the more flexible region of the tissue traction device; and the method further includes selectively delivering inflation medium through the flexible tubular element to control bending of the tissue traction device toward the stiffening element

In some aspects, a method for applying traction to a target tissue includes engaging a first tissue-engagement feature of a tissue traction device with the target tissue; engaging a second tissue-engagement feature of the tissue traction device with tissue spaced apart from the target tissue; and actuating the tissue traction device to bend along a flexible segment thereof which is more flexible than surrounding regions, towards a region of the tissue traction device opposite the flexible region to cause the tissue traction device to apply traction to the target tissue.

In some embodiments, the tissue traction device is tubular, and the method further includes actuating the tissue traction device by delivering an inflation medium to a lumen defined within the tissue traction device to expand the flexible segment of the tissue traction device relative to surrounding regions to cause the tissue traction device to bend in a direction generally opposite the flexible segment. In some embodiments, the tissue traction device includes a plurality of flexible tubular elements coupled together along a common join region; the flexible segment of the tissue traction device is formed along a segment of each of the plurality of flexible tubular elements generally opposite the common join region; and the method further includes selectively actuating one or more of the plurality of flexible tubular elements of the tissue traction device to control bending of the actuated flexible tubular element towards the common join region and to control bending of the tissue traction device in more than one direction. In some embodiments, each flexible tubular element is formed by a distal segment of a flexible tubular element forming a tissue traction system, each flexible tubular element having a lumen for delivering inflation medium to the tissue traction device and a sealed distal end, and the method further includes selectively delivering inflation medium through one or more of the flexible tubular elements to control bending of the tissue traction device in more than one direction. In some embodiments, the flexible tubular element of the tissue traction device is formed along a distal segment of a flexible tubular element having a lumen for delivering inflation medium to the tissue traction device and a sealed distal end; a stiffening element is applied along a limited region about the perimeter of the flexible tubular element of the tissue traction device so that surrounding regions define the more flexible region of the tissue traction device; and the method further includes selectively delivering inflation medium through the flexible tubular element to control bending of the tissue traction device toward the stiffening element.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue traction system and tissue traction device formed in accordance with various principles of the present disclosure.

FIG. 2 illustrates a cross-sectional view of a portion of the tissue traction device of FIG. 1 along line II-II.

FIG. 3 illustrates a perspective view of an example of an embodiment of a tissue traction system and actuation mechanism in accordance with various principles of the present disclosure.

FIG. 4 illustrates operation of an example of an embodiment of a tissue traction system such as illustrated in FIGS. 1-3.

FIG. 5 illustrates operation of an example of an embodiment of a tissue traction system such as illustrated in FIGS. 1-3.

FIG. 6 illustrates a perspective view of another example of an embodiment of a tissue traction system and tissue traction device formed in accordance with various principles of the present disclosure.

FIG. 7 illustrates operation of an example of an embodiment of a tissue traction system such as illustrated in FIG. 6.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “channel” or “bore” is not limited to a circular cross-section. As used herein, a “free end” of an element is a terminal end at which such element does not extend beyond.

In accordance with various principles of the present disclosure, a tissue traction system and a tissue traction device are configured and designed to apply a traction force to (e.g., apply tension to, such as to retract) a segment of tissue separated (e.g., dissected) from surrounding tissue at a treatment site. It will be appreciated that terms such as segment, portion, area, section, etc., may be used interchangeably herein without intent to limit, reference generally being made to a section as a general region, and a segment as a particular part, for the sake of convenient differentiation and without intent to limit. For the sake of convenience and without intent to limit, the separated segment of tissue may be referenced herein as a tissue flap. Further, it will be appreciated that the treatment site may be referenced alternatively herein as an anatomical site, delivery site, deployment site, implant/implantation site, target site, etc., without intent to limit.

More particularly, in accordance with various principles of the present disclosure, a tissue traction device is configured to bend in a desired (e.g., predetermined) direction to apply traction to a tissue flap to which the tissue traction device is operatively engaged. It will be appreciated that terms such as engage (and other grammatical forms thereof) may be used interchangeably herein with terms such as couple, grasp, hold, clasp, clip, anchor, associate, attach, affix, secure, etc. (and other grammatical forms thereof), without intent to limit. In some embodiments, the tissue traction device has an elongate cylindrical body with a generally cylindrical surface. One or more tissue-engagement features may be operatively associated with the tissue traction device, such as extending along a surface thereof. In some embodiments, a tissue-engagement feature is configured to be operatively engaged by a tissue-engagement member. In some embodiments, a tissue-engagement feature is an extension such as a loop engageable by a separately-formed tissue-engagement member such as a tissue clip configured to operatively engage the tissue traction device with tissue at a treatment site. In some embodiments, a tissue-engagement feature is formed with a tissue-engagement member, such as a clip or jaws configured to grasp tissue, already coupled thereto.

The tissue traction system may be used to actuate the tissue traction device to bend in one or more different directions. In some embodiments, the tissue traction system includes one or more elongate elements coupled to the tissue traction device to actuate movement of the tissue traction device. In some embodiments, the tissue traction device includes a region which is more flexible than surrounding regions such that actuation of the tissue traction device causes the tissue traction device to bend along the more flexible region in a direction of a less flexible surrounding region (generally towards a region substantially diametrically opposite the more flexible region). In some embodiments, only a limited perimetral segment (a segment defined along the surface of the tissue traction device and bound by longitudinally extending boundaries perimetrically/circumferentially spaced apart from each other) of the tissue traction device has increased flexibility and/or expandability relative to surrounding regions. As such, with only a portion of a perimeter of the tissue traction device flexing to a greater degree than surrounding regions of the tissue traction device, the tissue traction device will bend at such flexible region, generally towards an opposite side of the tissue traction device which is less flexible. The flexible region may extend along only a limited longitudinal extent of the tissue traction device or along the entire length of the tissue traction device. The flexible region may be a limited pre-stretched region and/or may be formed from a different material than surrounding less flexible region, and/or may have a thinner wall thickness than surrounding regions to allow for relatively increased flexibility and/or otherwise formed asymmetrically about the perimeter thereof to allow flexing of the tissue traction device to be directed in a particular direction. It will be appreciated that the flexible segment may extend along only a limited longitudinal extent of the tissue traction device or may be along the entire length of the tissue traction device.

In some embodiments, the traction device comprises at least one flexible tubular element. The flexible segment of such tissue traction device may additionally or alternatively be more expandable than other regions of the tissue traction device. Application of an inflation medium into the flexible tubular element of the tissue traction device causes the flexible tubular element to expand and bend along the flexible and/or expandable region, thereby applying a traction force to tissue to which the traction device is coupled.

In some embodiments, the tissue traction system and the tissue traction device apply and maintain traction on a tissue flap using pneumatic or hydraulics force. For example, the tissue traction system may be configured to deliver an inflation medium to an inflation lumen within a flexible tubular element of the tissue traction device to cause the tissue traction device to expand along a flexible and/or expandable region thereof and thus to bend towards a less-expandable region (e.g., opposite the flexible and/or expandable region) to apply traction to a tissue flap. In some embodiments, the flexible tubular element has a flexible expandable region surrounded by less flexible regions. Application of an inflation medium into a flexible tubular element of the tissue traction device causes the flexible expandable region of the flexible tubular element to expand. However, the less flexible regions do not expand (or may expand, but significantly less than the flexible expandable region expands). As a result, the flexible tubular element of the tissue traction device bends towards the less flexible regions as the more flexible region increases in size/length (and forms the outer part of the bent tube with the larger radius of curvature).

In some embodiments, the tissue traction system includes one or more elongate elements operatively coupled with the tissue traction device to actuate the tissue traction device to cause the tissue traction device to bend in the desired direction. In some embodiments, the tissue traction system and device use one or more tubular elements. The tubular elements may be configured at their distal end regions (at the distal end and a limited longitudinal extent proximal thereto) to form the traction device which is coupled to tissue to which traction is to be applied. For instance, only a limited region of a distal end of each of the one or more tubular elements of the tissue traction system is configured to form the tissue traction device (e.g., a flexible tubular element of the tissue traction device). Moreover, to allow directionality of the resulting bending of the tissue traction device upon actuation by the tissue traction system (e.g., application and/or delivery of an actuation medium to the tissue traction device), only a limited region along the perimeter of the distal end region of each of the one or more tubular elements of the tissue traction system (forming the tissue traction device) is less flexible. In other words, only a limited portion of a tubular element at the distal end thereof (the distal end region forming the tissue traction device) has a region about a limited extent about the perimeter of the tubular element with a greater flexibility and/or expandability than the remaining regions of the tubular element. As such, with only a limited region along the perimeter around the tissue traction device flexing or expanding to a greater degree than surrounding regions, the tissue traction device will bend at such flexible expandable region, generally towards an opposite side of the tissue traction device which is less flexible and/or expandable. The flexible expandable region may be a limited pre-stretched region and/or formed from a different material than surrounding less flexible and/or expandable regions, and/or have a thinner wall thickness than surrounding regions to allow for relatively increased flexibility and expandability and/or otherwise formed asymmetrically about the perimeter thereof to allow flexing to be directed in a particular direction.

It will be appreciated that instead of a limited region, along a limited extent about the perimeter of a tissue traction device, having increased flexibility and/or expandability, the tubular element forming the tissue traction device may be formed of a material with substantially uniform properties along the length and perimeter thereof, with a limited region along a limited extent about the perimeter of a tissue traction device having increased rigidity relative to the flexibility of the tubular element. As such, the portion of the flexible tubular element forming the tissue traction device expands to a greater extent in regions surrounding the region with increased rigidity, and therefore bends the tissue traction device towards the region with increased rigidity. In some embodiments, the tissue traction device is formed along a distal region of a flexible tubular element of the tissue traction system, and a increased rigidity/rigid section is formed along a limited region along a limited extent about the perimeter of the tissue traction device (i.e., along only one side of or vertical segment of the surface of the tissue traction device). For instance, a stiffening element may be applied longitudinally along the tissue traction device along a limited peripheral extent of the tissue traction device. The rigid element may be a material with reduced flexibility or expandability relative to the tubular element forming the tissue traction device, or otherwise may simply add wall thickness thereto. The stiffening element may be a material or coating applied to a limited region of the tube wall to rigidify or otherwise reduce the expandability such region.

Various embodiments of tissue traction systems and tissue traction devices will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by 100 and redundant description is generally omitted for the sake of brevity. Moreover, certain features in one embodiment may be used across different embodiments and are not necessarily individually labeled when appearing in different embodiments. Finally, it will be appreciated that relative proportions of elements of the illustrate system and device and the environment in which the system and device are illustrated are not necessarily drawn to scale.

Turning now to the drawings, FIG. 1 illustrates an example of an embodiment of a tissue traction system 100 with one or more elongate elements 110 forming a tissue traction system 100 that can apply force to tissue, such as traction force for lifting the tissue. In the illustrated example of an embodiment of a tissue traction system 100, the tissue traction device 120 is defined by a longitudinal extent of the elongate elements 110 extending a desired distance proximally from the distal ends 111 of the elongate elements 110 to define a length L of the tissue traction device 120 along a longitudinal axis LA. For instance, when used in gastrointestinal system, the distance should be enough to reach the stomach walls or the intestines wall, depending on the particular location of the tissue traction device 120. However, other configurations are within the scope and spirit of the present disclosure.

In accordance with various principles of the present disclosure, is an elongate cylindrical body with a segment 124 thereof configured to be more flexible and/or expandable than surrounding regions of the tissue traction device 120. Such segment 124 may be along a limited circumferential/perimetrical extent about the circumference/perimeter of the tissue traction device 120 to allow flexing or bending in a controllable and predictable direction. It will be appreciated that terms such as flexing, bending, navigating, steering, moving, etc., (including other grammatical forms thereof) are used interchangeably herein without intent to limit. Moreover, it will be appreciated that the cylindrical body of the tissue traction device 120 may have a circular cross-section and thus a circumference, or may have cross-sections of different shapes and thus reference may be made more generally to a perimeter thereof. Reference herein to a circumference or perimeter may be for the sake of convenience and should not be understood as limiting the cross-section of the tissue traction device 120 to a particular shape. Typically, the flexible and/or expandable segment 124 is defined by the tissue traction device 120 and thus is limited in its longitudinal extent to the length L of the tissue traction device 120. However, it will be appreciated flexible and/or expandable segment 124 may extend along only a limited longitudinal extent of the tissue traction device 120 (not along the entire length L).

More particularly, in accordance with various principles of the present disclosure, the above-described flexible and/or expandable segment 124 is a longitudinal circumferential segment 124 of the tissue traction device 120 formed to have increased flexibility and/or expandability relative to surrounding regions of the tissue traction device 120. For instance, the flexible and/or expandable segment 124 may be a limited pre-stretched region (e.g., causing the region to be able to stretch to a greater extent than surrounding regions) and/or may be formed from a different material than surrounding regions, and/or may have a thinner wall thickness than surrounding regions to allow for increased flexibility and expandability relative to surrounding regions along the perimeter of the tissue traction device 120. In some embodiments, such flexible and/or expandable segment 124 is formed asymmetrically about the perimeter of the tissue traction device 120 to allow directional flexing of the tissue traction device 120 (i.e., to cause flexing of the tissue traction device 120 to be directed in a particular direction, generally opposite, such as diametrically opposite, the flexible and/or expandable segment 124). At least in the example of an embodiment illustrated in FIG. 1, the flexible and/or expandable segment 124 is a longitudinal circumferential segment 124 extending longitudinally along at least a portion of the length L of the tissue traction device 120, with longitudinal boundaries circumferentially spaced apart from each other. In other words, the longitudinal circumferential segment 124 extends along only a portion of a side of a longitudinal extent of the tissue traction device 120. In some embodiments, the longitudinal extent L of the longitudinal circumferential segment 124 is along the entire length L of the tissue traction device 120. However, it will be appreciated that the longitudinal circumferential segment 124 may be shorter than the full length L of the tissue traction device 120, strategically positioned to cause bending of the tissue traction device 120 at a desired area along the length L of the tissue traction device 120. For the sake of convenience and without intent to limit, the flexible/expandable segment/longitudinal circumferential segment 124 may be referenced herein as a flexible and/or expandable bending region 124 of the tissue traction device 120.

Additionally or alternatively, regions of the tissue traction device 120 surrounding the flexible and/or expandable bending region 124 are formed to have reduced flexibility and/or expandability relative to the flexible and/or expandable bending region 124. Because the flexible and/or expandable bending region 124 does not extend along the entire perimeter or circumference of the tissue traction device 120, actuation thereof (e.g., expansion) causes the tissue traction device 120 to bend in the area of the flexible and/or expandable bending region 124 towards less flexible and/or less expandable regions, as described in further detail below.

In accordance with various principles of the present disclosure, the one or more elongate elements 110 of the tissue traction system 100 are one or more flexible tubular elements 110 defining the tissue traction device 120 along a distal length L of the flexible tubular elements 110. The distal ends 111 of the flexible tubular elements 110 are sealed so that an actuation medium may be delivered by the tissue traction system 100 to the tissue traction device 120 to actuate the tissue traction device 120. In some embodiments, the actuation medium is an inflation medium. The flexible/expandable bending region 124 is configured to expand to a greater extent than surrounding regions of the tissue traction device 120 upon delivery of inflation medium into the tissue traction device 120. The expansion of the flexible/expandable bending region 124 increases the length of such region/segment of the tissue traction device 120 relative to the length of surrounding segments/regions, thereby causing bending of the tissue traction device 120 towards the less flexible/expandable regions of the tissue traction device 120 (generally opposite, such as diametrically opposite the flexible and/or expandable bending region 124). The flexible/expandable bending region 124 defines the outer bend of the tissue traction device 120 or, in other words, is positioned along the bent surface of the tissue traction device 120 with the larger radius of curvature (resulting from actuation of the tissue traction system 100 bending the tissue traction device 120).

In some embodiments, a tissue-engagement feature 122a is provided along the distal end 121 of the tissue traction device 120. For instance, if a plurality of flexible elements 110 form the tissue traction device 120, a tissue-engagement feature 122a may be provided along a distal end 111 of each of the flexible elements 110. The tissue-engagement feature 122a may be a projection which facilitates attachment of the tissue traction device 120 to tissue, such as with a tissue-engagement member. In the example of an embodiment illustrated in FIG. 1, the tissue-engagement feature 122a is a loop to which a separately-formed tissue-engagement member 130 is engaged. The example of an embodiment of a tissue-engagement member 130 illustrated in FIG. 1 has one or more jaws 132 configured to grasp tissue at the treatment site TS, such as target tissue TT to which traction is to be applied by the tissue traction device 120 and the tissue traction system 100. However, it will be appreciated that other forms of tissue-engagement members such as known to those of ordinary skill in the art are within the scope and spirit of the present disclosure. Moreover, it will be appreciated that the tissue-engagement member may be formed integrally with the tissue-engagement feature 122a instead of being a separately-formed element coupled to the tissue-engagement feature 122a. The tissue-engagement member 130 may be coupled or provided with the tissue-engagement feature 122a during delivery of the tissue traction device 120 to the treatment site TS. The tissue-engagement member 130 may be actuated to grasp tissue at the treatment site TS to deploy/couple the tissue traction device 120 to the treatment site TS. Any desired actuator or end effector such as known to those of ordinary skill in the art (the details of which are not critical to the present disclosure) may be used to actuate the tissue-engagement member 130.

In some embodiments, one or more additional tissue-engagement features, such as a proximal tissue-engagement feature 122b, as illustrated in FIG. 1, are provided along a proximal end 123 of the tissue traction device 120. Such additional tissue-engagement features may be similar in form to the tissue-engagement feature 122a described above, reference being made to such description of the sake of brevity, or other configurations facilitating engagement of the tissue traction device 120 with tissue. The proximal tissue-engagement feature 122b illustrated in FIG. 1 is similar to the distal tissue-engagement feature 122a described above. Typically, however, a tissue-engagement member 130 is not engaged with the proximal tissue-engagement feature 122b during delivery of the tissue traction device 120 to the treatment site TS. Instead, a tissue-engagement member 130 typically is separately delivered and is actuated to grasp the proximal tissue-engagement feature 122b and then to grasp tissue spaced apart from the target tissue TT to apply traction to the target tissue TT in a manner understood by those of ordinary skill in the art. Further details of an example of a use of a tissue traction system 100 and tissue traction device 120 as illustrated in FIG. 1 are provided below.

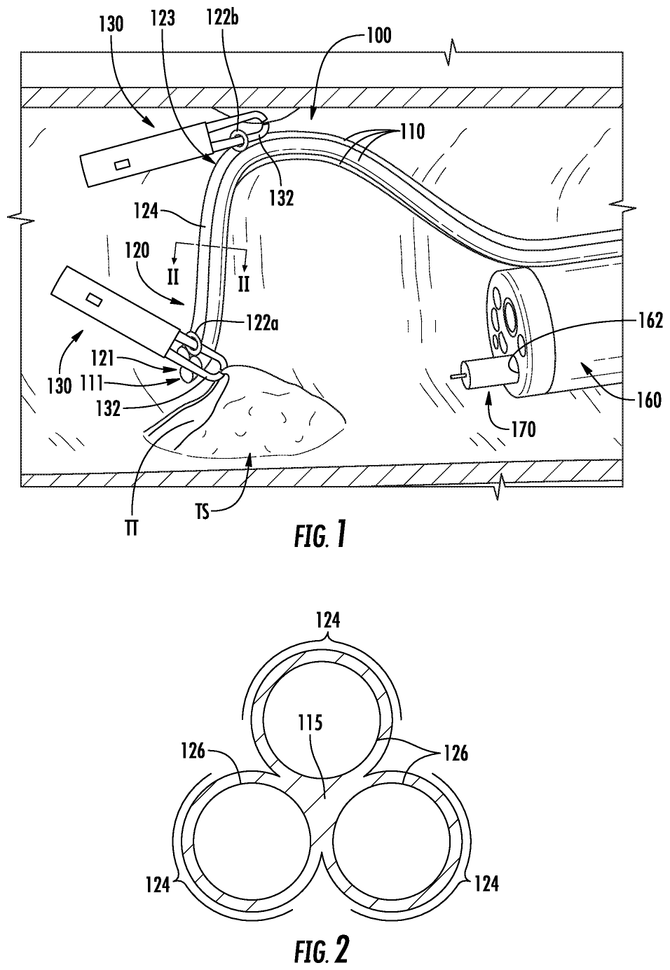

As shown in FIG. 2, illustrating a cross-sectional view along line II-II of FIG. 1, the illustrated example of an embodiment of a tissue traction device 120 of FIG. 1 may be formed with one or more flexible tubular elements 126. Each of the flexible tubular elements 126 may be formed along a respective flexible tubular element 110 forming the tissue traction system 100. More specifically, the flexible tubular elements 126 may be formed along a distal segment of the flexible tubular elements 110 of the tissue traction system 100 extending proximally from respective distal ends 111 thereof. In the illustrated example of an embodiment, the tissue traction device 120 is formed with three flexible tubular elements 126 arranged in a generally triangular formation to be conjoined (or at least positioned adjacent if not joined) along a common join region 115. The common join region 115 generally inhibits expandability of the walls of the flexible tubular elements 126 in such region. As such, the regions surrounding the common join region 115 form the flexible and/or expandable bending region 124 of the flexible tubular elements 126 of the tissue traction device 120. In some embodiments, the flexible tubular elements 126 are formed of an elastic material (e.g., silicone or other suitable biocompatible material) allowing radial expansion thereof at least along the flexible and/or expandable bending region 124. In some embodiments, the flexible and/or expandable bending region 124 is pre-stretched along a defined length in order to further facilitate expansion thereof and to allow the tissue traction device 120 to reach the walls of tissue at which a procedure is to be performed.

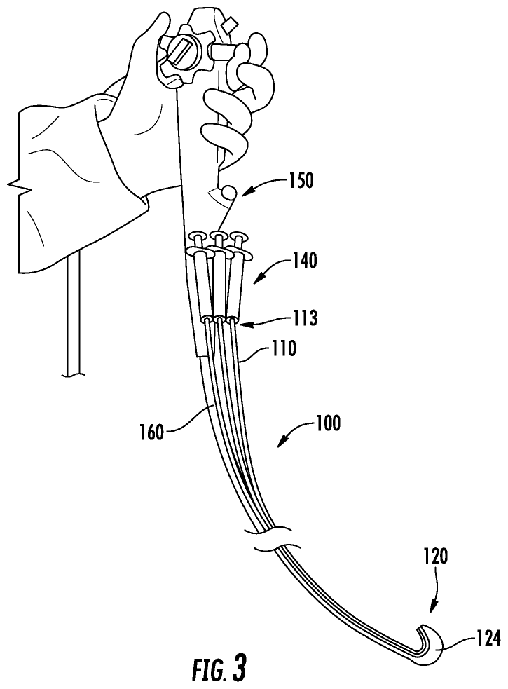

In accordance with various principles of the present disclosure, one manner in which the tissue traction device 120 is actuated to bend (such as illustrated in FIG. 1) is by expansion of a flexible and/or expandable bending region 124 of the tissue traction device 120 caused by selective inflation of the distal portions of the flexible tubular elements 110 forming the tissue traction device 120. For instance, as illustrated in FIG. 3, one or more actuation mechanisms 140 are operatively coupled with respective proximal ends 113 of flexible tubular elements 110 of a tissue traction system 100 such as in FIG. 1 and FIG. 2. In some embodiments, the actuation mechanisms 140 are syringes which apply an actuation medium, such as an inflation medium (e.g., saline or other biocompatible fluid, such as carbon dioxide gas), into the tissue traction device 120 via the flexible tubular elements 110. The actuation mechanisms 140 are shown alongside a separately-formed control handle 150. However, other arrangements are within the scope and spirit of the present disclosure. The control handle 150 may be used to control an endoscope 160 with which instruments, devices, tools, etc. (such terms being used interchangeably herein without intent to limit) may be delivered to the treatment site TS to perform the intended procedure at the treatment site TS. For instance, as illustrated in FIG. 1, the endoscope 160 may deliver a cutting tool 170 (e.g., a knife, scalpel, electrocautery device, etc., the present disclosure not being limited to a particular such tool) to perform a tissue dissection or resection, with the tissue traction device 120 applying traction to the flap of tissue thereby created. In the example of an embodiment illustrated in FIG. 1 and FIG. 3, the tissue traction system 100 is configured to extend external to the endoscope 160, instead of through a working channel 162 of the endoscope 160. Such configuration frees the working channel 162 of the endoscope 160 for delivery of a cutting tool 170 while the tissue traction system 100 is in place and operable. The tissue traction system 100 may be coupled to the endoscope 160. The tissue traction system 100 may be freely movable with respect to the endoscope 160, whether or not coupled to the endoscope 160.

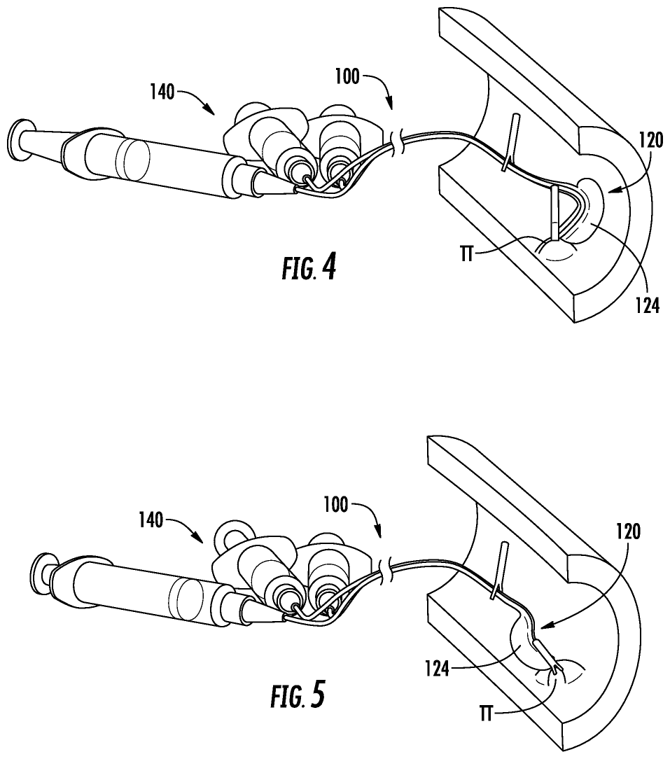

As illustrated in FIG. 3, selective application of actuation medium, such as controlled by one of the actuation mechanisms 140, causes expansion of only the flexible and/or expandable bending region 124 in fluid communication with the flexible tubular element 110 operatively associated with the actuated actuation mechanism 140. For instance, application of actuation medium to a selected flexible tubular element 126 of the tissue traction device 120 increases pressure within such flexible tubular element to cause expansion of the flexible and/or expandable bending region 124. The regions of the walls of the flexible tubular elements 126 of the tissue traction device 120 along the common join region 115 are less apt to expand (e.g., may be more rigid, or at least do not have any space to which they can expand). Thus, expansion of the flexible and/or expandable bending region 124 of the actuated flexible tubular element 126 of the tissue traction device 120 causes the actuated flexible tubular element 126 to bend towards the common join region 115 (e.g., by effectively increasing the length of the flexible and/or expandable bending region 124 without equally increasing the length of the common join region 115 of the actuated flexible tubular element). The medical professional operating the tissue traction system 100 can decide which of the flexible tubular elements 126 of the tissue traction system 100 and tissue traction device 120 to actuate (e.g., pressurize) to direct bending of the tissue traction device 120 in the desired direction. For instance, the medical professional can actuate a selected actuation mechanism 140 to inflate one or the flexible tubular elements 126 forming the tissue traction device 120 to cause such actuated flexible tubular element 126 to bend and thereby to cause the tissue traction device 120 to bend to lift the target tissue TT in different directions or along different angles (e.g., along different force vectors), such as illustrated in FIG. 4 and FIG. 5.

An example of operation of a tissue traction system 100 and tissue traction device 120 as illustrated in FIG. 1, FIG. 2, FIG. 3, FIG. 4, and FIG. 5 is as follows. A tissue-engagement member 130 is introduced into the endoscope 160 and the tissue-engagement member 130 is attached to the tissue-engagement feature 122a at the distal end 121 of the tissue traction device 120. Optionally, though not necessarily, the tissue-engagement member 130 is introduced into the endoscope 160 with the endoscope 160 outside the patient. The endoscope 160 is introduced into the patient and the tissue-engagement member 130 (operatively engaged with the tissue-engagement feature 122a on the tissue traction device 120) is attached to the target tissue TT. A second tissue-engagement member 130 is introduced through the working channel 162 of the endoscope 160. The medical professional picks one of the additional tissue-engagement features, such as the illustrated proximal tissue-engagement feature 122b, and attaches the second tissue-engagement member 130 thereto and to tissue spaced apart from the target tissue TT (e.g., on an opposite side of a lumen wall in which the target tissue TT is located). The medical professional controls the position of the target tissue TT using the actuation mechanisms 140 in a manner as described above.

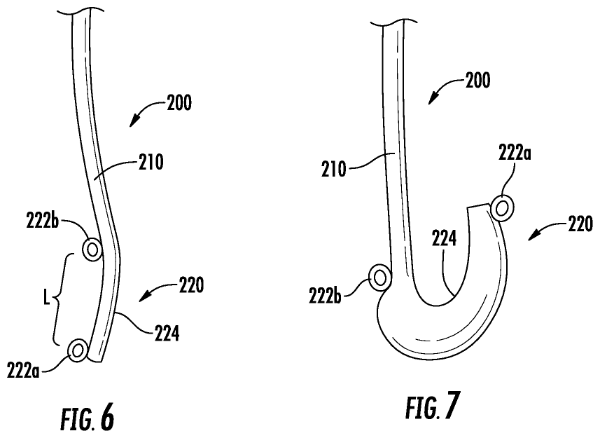

It will be appreciated that various modifications to the above-described tissue traction system 100 and/or tissue traction device 120 are within the scope and spirit of the present disclosure. For instance, only one elastic flexible tubular element may be used in the tissue traction system to form the tissue traction device. In such embodiment, as illustrated in FIG. 7 and FIG. 8, a limited region of the tissue traction device 220 formed by a single flexible tubular element 210 of the illustrated example of an embodiment of a tissue traction system 200 may be less flexible to provide similar bending action as described above with reference to the tissue traction system 100 and tissue traction device 120 illustrated in FIGS. 1-5, For instance, a longitudinal circumferential segment 224, along only a portion of the perimeter or circumference or face of the flexible tubular element 210, is made less flexible/more rigid and/or less expandable than the other surrounding regions. As with the example of an embodiment described above, the longitudinal circumferential segment may extend along the entire length L or only a limited extent along the length L of the tissue traction device 220. The limited less flexible and/or less expandable bending segment 224 may have a similar effect as the flexible and/or expandable bending segment 124 of the tissue traction device 120 of the above-described example of an embodiment of a tissue traction system 100, allowing bending of the tissue traction device 220 in only one direction, towards the less-flexible and/or less expandable bending segment 224. In some embodiments, the less-flexible and/or less expandable bending segment 224 is formed by application of a stiffening element, such as additional material (a patch or coating or the like), along a limited extent about the perimeter of the tissue traction device 220 and optionally along a limited length of the tissue traction device 220. This solution thus may be simpler than the previously-described embodiment, though may lack some steerability options available to the previously-described embodiment. It will be appreciated that the example of an embodiment illustrated in FIG. 6 and FIG. 7 has various other features in common with the example of an embodiment illustrated in FIGS. 1-5. Accordingly, the common features are identified by common reference elements increased by 100, and, for the sake of brevity, the descriptions of the common features will not be repeated. For purposes of clarity, not all components having the same reference number are numbered.

A tissue traction system 200 such as illustrated in FIG. 6 and FIG. 7 may be operated in a similar manner as described above with respect to the tissue traction system 100 of FIGS. 1-5, and reference is thus made to the above descriptions of examples of operation for the sake of brevity and without intent to limit. It will be appreciated that in various embodiments, some steps of assembling a tissue retraction device may occur outside of the patient's body, while other steps involved in assembling the tissue retraction device may occur within the patient. The steps described herein do not necessarily occur in a specific order and/or timing.

It will be appreciated that further modifications to the above-described tissue traction systems and tissue traction devices are within the scope and spirit of the present disclosure. For instance, a tissue traction device formed in accordance with various principles of the present disclosure need not be coextensive with the elongate elements forming a tissue traction system formed in accordance with various principles of the present disclosure. For instance, the tissue traction device may be formed from flexible tubular elements separately formed from the elongate elements forming the tissue traction system. As such, the diameters of the elongate elements forming the tissue traction system may be smaller than the diameters of the components of the tissue traction device to facilitate transluminal/transcatheter navigation of the tissue traction system. Such formation may facilitate formation of a tissue traction device with a segment of increased flexibility and/or rigidity.

Various further benefits of the various aspects, features, components, and structures of a tissue traction system and/or tissue traction device such as described and discussed above may be appreciated by those of ordinary skill in the art. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will be appreciated features described with respect to one embodiment typically may be applied to another embodiment, whether or not explicitly indicated. The various features hereinafter described may be used singly or in any combination thereof. Therefore, the present invention is not limited to only the embodiments specifically described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue traction system comprising:
a tissue traction device; and
one or more elongate elements operatively coupled with said tissue traction device to actuate bending of said tissue traction device;
wherein a longitudinal circumferential region of said tissue traction device is more flexible than surrounding regions of said tissue traction device so that actuation of said tissue traction device causes bending along the more flexible region toward a less flexible region.

2. The tissue traction system of claim 1, wherein said tissue traction device is formed by a distal extent of said one or more elongate elements extending a length proximal to distal ends of said one or more elongate elements.

3. The tissue traction system of claim 2, wherein at least one of said elongate elements is a flexible tubular element.

4. The tissue traction system of claim 3, wherein said at least one flexible tubular element has a sealed distal end.

5. The tissue traction system of claim 4, further comprising an actuation mechanism operatively coupled to said one or more elongate elements to cause bending of said tissue traction device.

6. The tissue traction system of claim 5 wherein:
a distal extent of said at least one flexible tubular element forms a flexible tubular element of said tissue traction device;
said longitudinal circumferential region of said tissue traction device is formed along a distal region of said at least one flexible tubular element and is more expandable than surrounding regions of said tissue traction device; and
said actuation mechanism delivers an inflation medium through said at least one flexible tubular element to said tissue traction device to expand said longitudinal circumferential region of said tissue traction device relative to surrounding regions of said tissue traction device to cause said tissue traction device to bend.

7. The tissue traction system of claim 1, further comprising an actuation mechanism operatively coupled to said one or more elongate elements to cause bending of said tissue traction device.

8. The tissue traction system of claim 1, wherein a stiffening element is applied along a limited region along the perimeter of said tissue traction device, resulting in surrounding regions being relatively more flexible.

9. The tissue traction system of claim 8, wherein:
said one or more elongate elements includes one flexible tubular element with a sealed distal end;
said tissue traction device is formed by a distal extent of said flexible tubular element extending a length proximal to the sealed distal end of said flexible tubular element; and
said tissue traction device is directionally inflatable upon application of an inflation medium thereto causing the more flexible region of said tissue traction device to expand and to bend said tissue traction device toward said stiffening element.

10. A tissue traction device comprising:
an elongate cylindrical body extending along a longitudinal axis and having a distal end and a proximal end and a flexible segment therebetween extending along only a portion of the perimeter of said cylindrical body;
wherein said flexible segment is more flexible than surrounding portions of said cylindrical body to facilitate bending of said cylindrical body along said flexible segment.

11. The tissue traction device of claim 10, wherein:
said cylindrical body is tubular and inflatable; and
said flexible segment is more expandable than surrounding portions of said cylindrical body so that inflation of said tubular cylindrical body causes expansion of said flexible segment and bending of said flexible segment towards an opposite, less expandable side of said cylindrical body.

12. The tissue traction device of claim 10, wherein said flexible segment extends only along a limited extent along the longitudinal axis of said cylindrical body.

13. The tissue traction device of claim 11, wherein said cylindrical body is formed of a flexible material and a stiffening element is applied to a limited extent around the perimeter of said flexible cylindrical body, said flexible segment being defined opposite said stiffening element.

14. The tissue traction device of claim 10, wherein said cylindrical body is formed of a flexible material and a stiffening element is applied to a limited extent around the perimeter of said flexible cylindrical body, said flexible segment being defined opposite said stiffening element.

15. The tissue traction device of claim 10, wherein said cylindrical body is a distal segment of a flexible tubular element of a tissue traction system.

16. A method for applying traction to a target tissue, said method comprising:
engaging a first tissue-engagement feature of a tissue traction device with the target tissue;
engaging a second tissue-engagement feature of the tissue traction device with tissue spaced apart from the target tissue; and
actuating the tissue traction device to bend along a flexible segment thereof which is more flexible than surrounding regions, towards a region of the tissue traction device opposite the flexible region to cause the tissue traction device to apply traction to the target tissue.

17. The method of claim 16, wherein the tissue traction device is tubular, said method further comprising actuating the tissue traction device by delivering an inflation medium to a lumen defined within the tissue traction device to expand the flexible segment of the tissue traction device relative to surrounding regions to cause the tissue traction device to bend in a direction generally opposite the flexible segment.

18. The method of claim 17, wherein:
the tissue traction device comprises a plurality of flexible tubular elements coupled together along a common join region;
the flexible segment of the tissue traction device is formed along a segment of each of the plurality of flexible tubular elements generally opposite the common join region; and
said method comprises selectively actuating one or more of the plurality of flexible tubular elements of the tissue traction device to control bending of the actuated flexible tubular element towards the common join region and to control bending of the tissue traction device in more than one direction.

19. The method of claim 18, wherein each flexible tubular element is formed by a distal segment of a flexible tubular element forming a tissue traction system, each flexible tubular element having a lumen for delivering inflation medium to the tissue traction device and a sealed distal end, said method further comprising selectively delivering inflation medium through one or more of the flexible tubular elements to control bending of the tissue traction device in more than one direction.

20. The method of claim 17, wherein:
the flexible tubular element of the tissue traction device is formed along a distal segment of a flexible tubular element having a lumen for delivering inflation medium to the tissue traction device and a sealed distal end;
a stiffening element is applied along a limited region about the perimeter of the flexible tubular element of the tissue traction device so that surrounding regions define the more flexible region of the tissue traction device; and
said method further comprising selectively delivering inflation medium through the flexible tubular element to control bending of the tissue traction device toward the stiffening element.
